(19) United States
(12) Patent Application Publication
(10) Pub. No.: US 2015/0173941 A1
(43) Pub. Date: Jun. 25, 2015

(54) FORCED AIR TEMPERATURE REGULATING PAD WITH TRIPLE-LAYER TECHNOLOGY

(71) Applicant: Brandon Cuongquoc Giap, San Diego, CA (US)

(72) Inventor: Brandon Cuongquoc Giap, San Diego, CA (US)

(21) Appl. No.: 14/574,722

(22) Filed: Dec. 18, 2014

Related U.S. Application Data
(60) Provisional application No. 61/918,668, filed on Dec. 20, 2013.

(51) Int. Cl. A61F 7/00

(52) U.S. Cl. CPC A61F 7/0085; A61F 7/0097; A61F 2007/0055

(57) ABSTRACT

A temperature regulating pad having an air-permeable top layer, a perforated middle layer, and a bottom layer. The perforated middle layer is located between the top layer and the bottom layer. The top layer and the perforated middle layer delimit an upper airspace and the perforated middle layer and the bottom layer delimit a lower airspace. Forced air is received into the lower airspace and flows from the lower airspace into the upper airspace through the perforated middle layer, exiting the air-permeable top layer into an external airspace the said top layer. The pad may be placed on an underlying support surface on which a patient rests.

### CROSS REFERENCE TO RELATED APPLICATIONS

This application also claims priority to U.S. Provisional Application 61/918,668 filed on Dec. 20, 2013.

### BACKGROUND

Hypothermia is a recognized common occurrence for patients during surgery. Patients who develop hypothermia are at a greater risk for complications, including a greater chance of heart problems, high rates of infection, increase blood loss and prolonged recovery. To counter this, medical personnel may cover the patient with blankets. Blankets are typically bulky, frequently unravel, and may fall off the patient during pre-surgery, surgery, post-surgery, or transport. Additionally, blankets may pose a safety risk and may interfere with the doctor or other personnel's ability to care for the patient. The use of forced warm air blankets or pads is known in the art; however, there are several downfalls. For example, these types of devices typically infuse predetermined high-temperature air through a bladder or pad to maintain the patient's body temperature in the normal range. The technology often utilizes a double layer or bladder type blanket made of a thin air proof bottom material in a perforated top layer. Warm air is forced into the bladder such that warm air is allowed to escape the perforated top layer and come in contact with the patient's skin area that is exposed to the blanket or pad. The shortcomings of this technology is that the tiny air jets coming out from the double layer blanket or pad can generate are relatively turbulent and, therefore, can cause the excitement of dust contained in or adjacent to the blanket or pad. This turbulent and potentially dusty air can compromise the otherwise engineered and sterile airflow created in the operating room. There are concerns, though not proven, of increased infection associated with the use of forced warm air technology using the traditional double layer or bladder technology as a result of the dust problem.

Accordingly, the present disclosure is directed to a temperature regulating pad or blanket that has reduced turbulence of air coming exiting the blanket or pad to the patient. Additionally, the temperature regulating pad or blanket is configured to generate an even air distribution (whether warm or cool) through the blanket toward the patient.

### BRIEF DESCRIPTION OF THE DRAWINGS

FIG. 1 is a perspective view of one embodiment of the temperature regulating pad.

FIG. 2 is a cross-sectional view of one embodiment of the temperature regulating pad.

FIG. 3 is a top view of the middle layer of the temperature regulating pad, showing the construction thereof.

FIG. 4 is a perspective view of one embodiment of the temperature regulating pad in an inflated state with forced warm air passing therethrough.

FIG. 5 is a cross-sectional view of one embodiment of the temperature regulating pad in an inflated state with forced warm air passing therethrough.

FIG. 6 A-C are an end views of one embodiment of the temperature regulating pad in an inflated state in use with a patient on surgical table, demonstrating the flow of warm air.

FIG. 7 is a perspective view of one embodiment of the temperature regulating pad in an inflated state in use with a patient on surgical table and attached to a warm air source.

### DETAILED DESCRIPTION

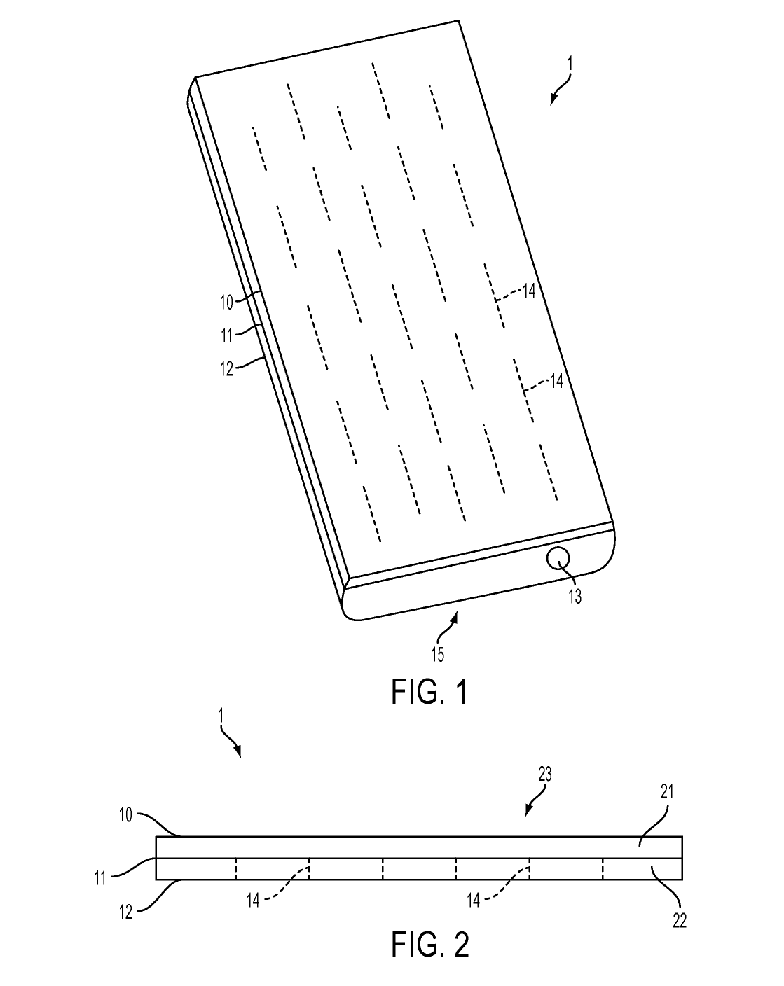

Shown in FIG. 1 is temperature regulating pad 1 generally configured as a planar element having a top layer 10, a middle layer 11, and a bottom layer 12. Temperature regulating pad 1 is shown at least partially transparent in FIG. 1 whereby a plurality of seal lines 14 attaching middle layer 11 to bottom layer 12 can be seen. Air inlet 13 may also be provided, in some embodiments, along the bottom edge 15 of the temperature regulating pad 1. The temperature regulating pad 1 may be configured as a standalone device to be placed on a stretcher, surgical table, bed, or other support surface. Alternatively, the temperature regulating pad 1 may be configured to be inserted into a sheet or pocket provided, for example, in the patient positioning device described in Applicant's patient positioning device invention described in U.S. application Ser. No. 14/340,611, U.S. application Ser. No. 13/359,734 and U.S. application Ser. No. 13/153,432.

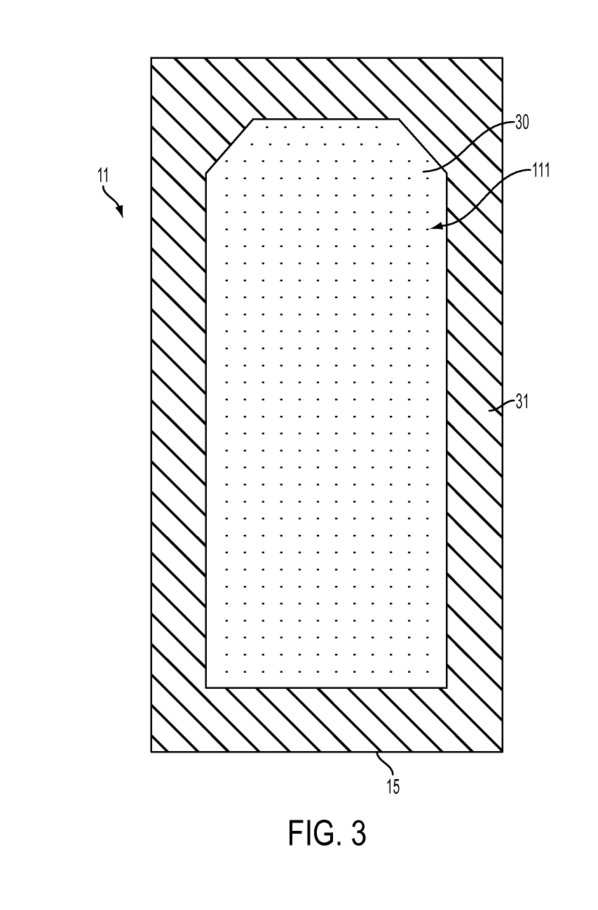

FIG. 2 shows a cross-section of the temperature regulating pad 1. As shown, middle layer 11 is disposed between bottom layer 12 and top layer 10. This construction divides the temperature regulating pad 1 into an upper airspace 21 delimited by the top layer 10 and middle layer 11, and a lower airspace 22 delimited by the middle layer 11 in the bottom layer 12. An external airspace 23 is located about and above top layer 10 and generally defines the area in which forced air is communicated to a patient resting on the temperature regulating pad 1. In some embodiments, a plurality of seal lines 14 attach the middle layer 11 to the bottom layer 12 at spaced apart intervals across the width of the temperature regulating pad 1. Referring back to FIG. 1, seal lines 14 are also disposed along the length of the pad 1 in spaced-apart fashion. In some embodiments, seal lines 14 are disposed in a matrix formation with respect to the surface of the temperature regulating pad 1. Air inlet 13 shown in FIG. 1 is located to be in flow communication with and introduce air into the lower airspace 22 and, therefore, is disposed between the middle layer 11 and bottom layer 12, in some cases along the bottom edge 15 thereof although other positions around the exterior perimeter of the temperature regulating pad 1 may be equally suitable.

In some embodiments, bottom layer 12 comprises a low friction nylon material and is substantially impermeable to air and is waterproof. In some embodiments, the middle layer 11 comprises a thin plastic sheet of which at least a portion of is perforated to include a plurality of apertures 111. In some embodiments the apertures 111 are dispersed evenly along at least a portion of the surface area of the middle layer 11. In some embodiments, the top layer 10 comprises an air-permeable, breathable but waterproof material, such as polypropylene, which will allow air to pass through from the upper airspace 21 to the external airspace 23 in order to warn a patient disposed on the temperature regulating pad 1.

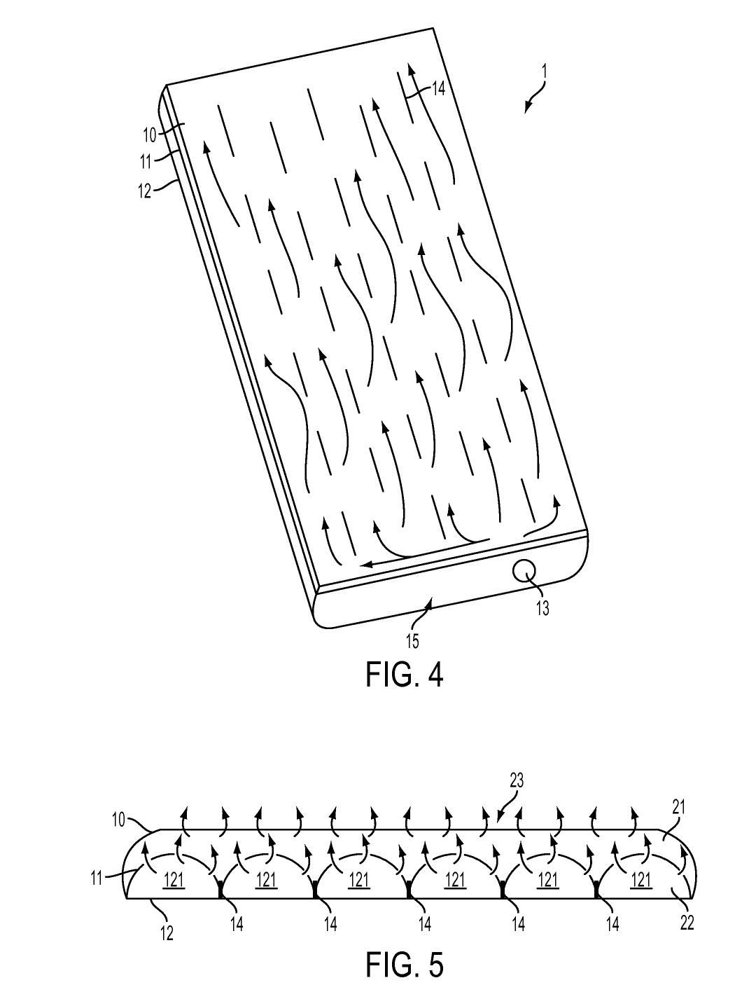

FIG. 3 is a top view of an embodiment of the middle layer 11 showing the construction thereof. In some embodiments, middle layer 11 includes a perforated center section 30 comprising the aforementioned perforated surface of the middle layer 11, which is surrounded by an impermeable border 31. As shown, the perforated center section 30 includes the plurality of apertures 111 disposed therethrough. The impermeable border 31 reinforces the construction of the temperature regulating pad 1 and also assures that air is forced up through the perforated center section 30 and does not get trapped at the terminal sides of the temperature regulating pad 1. The width of the impermeable border 31 can vary depending on the desired construction; however, it should be sufficiently wide such as to generally cause the majority of introduced air to pass through the perforated center section 30, rather than to get trapped at the edges of the temperature regulating pad 1.

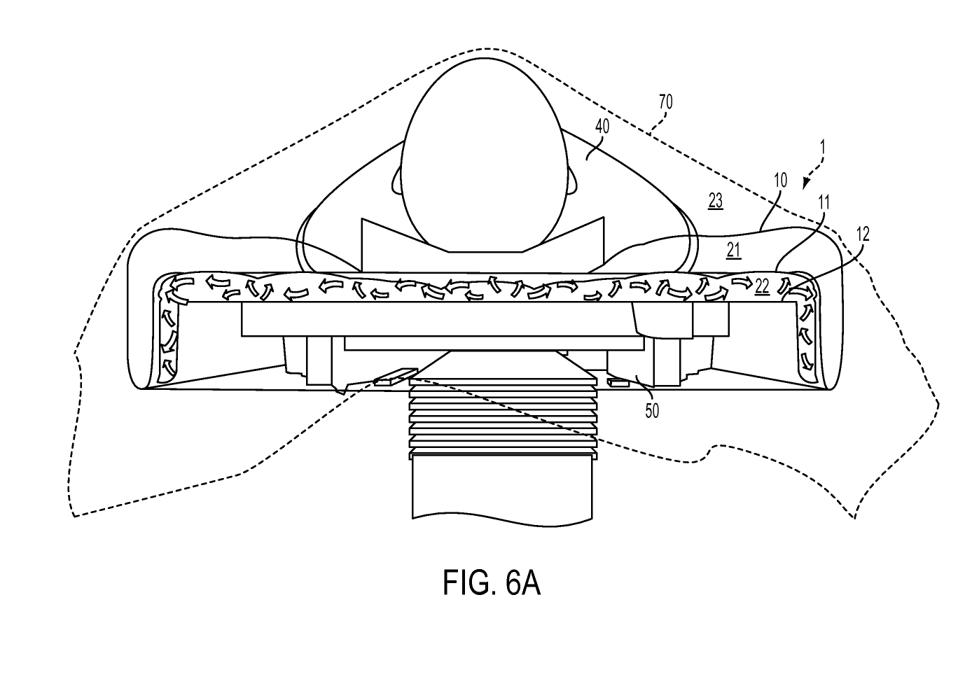

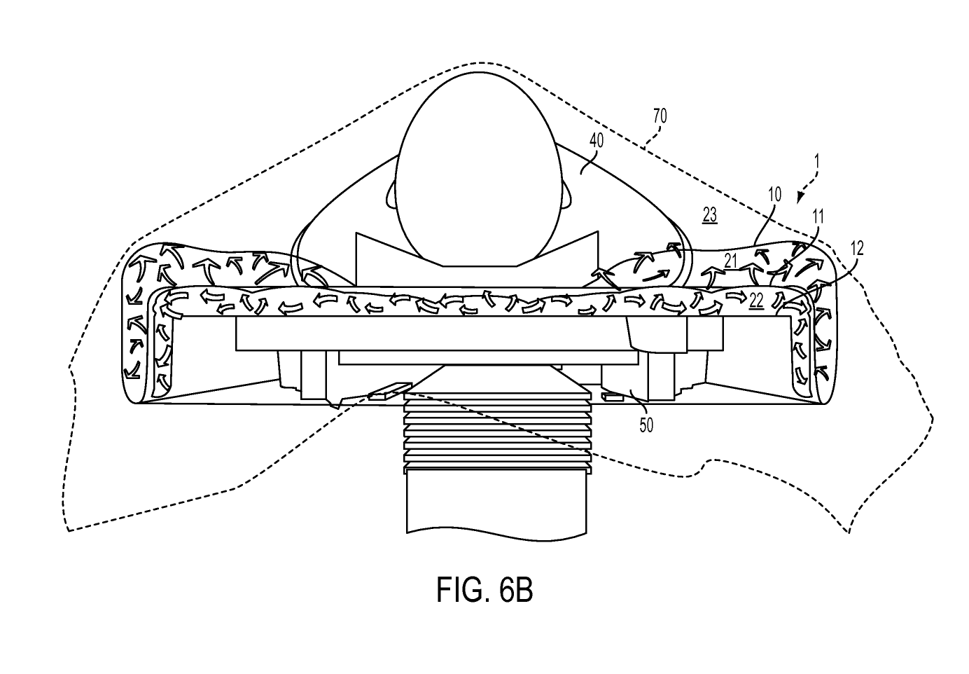

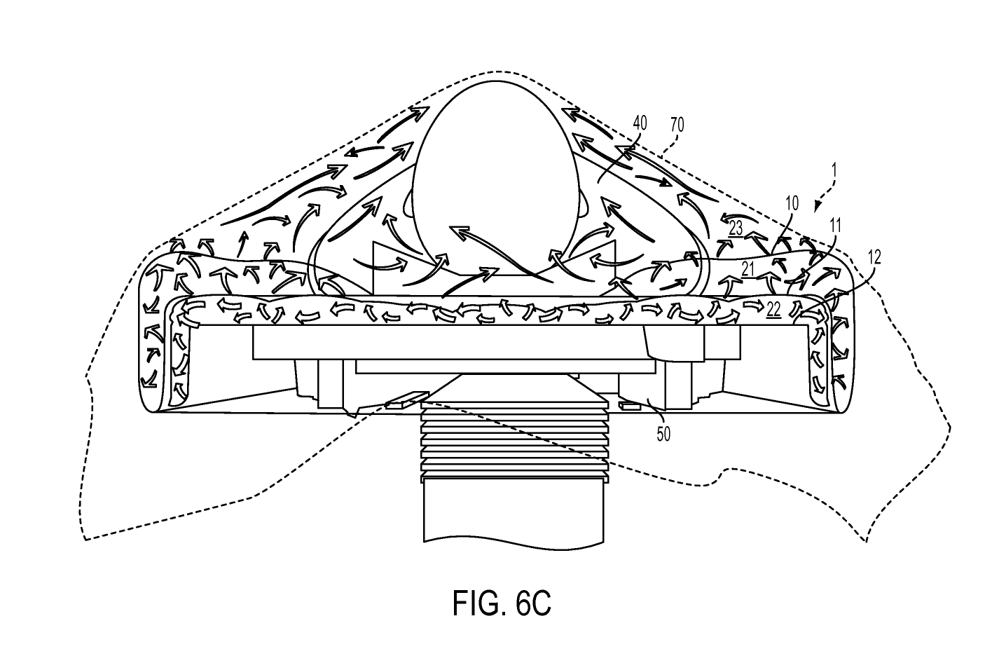

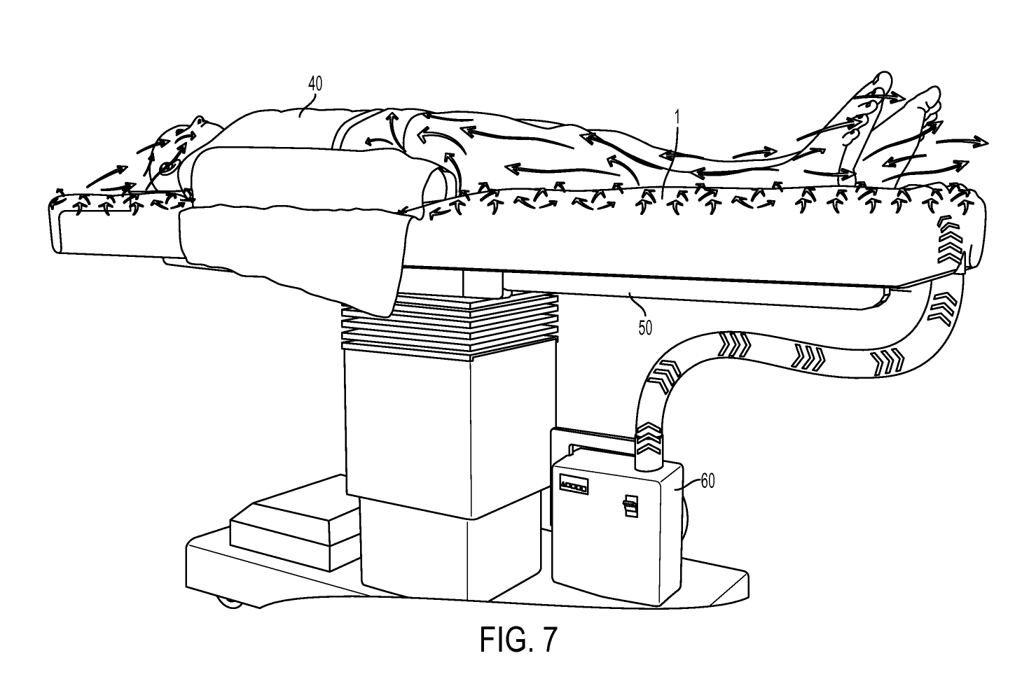

FIGS. 4 and 5 depict the temperature regulating pad 1 in an inflated or action state. FIGS. 6A-6C show various stages of the inflated state of the temperature regulating pad 1 from an end view in use with a patient 40 resting on an underlying support surface 50. FIG. 7 shows a perspective view of the temperature regulating pad 1 in use in conjunction with a forced air source 60. Air from an external source 60 is provided into lower airspace 22 through air inlet 13 at the bottom edge of the temperature regulating pad 1. In some embodiments, air inlet 13 is configured to receive a hose or tubing from a forced air machine such as a compressor or the like that provides an air source 60. As air is forced through air inlet 13, the temperature regulating pad 1 at least partially inflates forming channels 121 disposed lengthwise in lower airspace 22 which are delimited by the plurality of seal lines 14 attaching the middle layer 11 to the bottom layer 12 (FIG. 6A). In some embodiments this causes middle layer 11 to expand upward with respect to bottom layer 12 such that the channels 121 form a substantially cylindrical or semi-cylindrical profile as shown in FIG. 5. As these channels are formed, a pressure differential is created and air proceeds from lower airspace 22 through apertures 111 of middle layer 11 and into upper airspace 21 (FIG. 6B). The air continues through the air-permeable breathable surface of the top layer 10 and into the external airspace 23 surrounding the patient 40 (FIG. 6C). Accordingly, with a patient 40 disposed on top of top layer 10 as air is forced through the temperature regulating pad 1, air exits through the top layer to surround the patient to increase and/or maintain the patient's body temperature. As shown in FIG. 4, air will flow through the channels 121 up and through the top layer 10 in an upward and outward direction toward the patient. As shown in FIGS. 6A-6C, an optional oversheet 70 may be provided which at least partially surrounds the patient 40 and the external airspace 23. In some embodiments, the oversheet 70 may comprise a thin plastic or polypropylene material and may include adhesive portions and/or an adhesive border to secure the oversheet 70 to the sides or bottom of the underlying support surface 50.

With the air initially forced into lower airspace 22, the air pressure inside lower airspace 22 is greater than the pressure inside upper airspace 21. The air pressure inside upper airspace 21 is greater than that of external airspace 23. Thus, a pressure gradient or differential is created with higher pressure lower airspace 22 as compared to that of the external airspace 23. As the air enters inlet 13 and into lower airspace 22, channels 121 and then the air flows through the apertures 111 of the middle layer 11 and into upper airspace 21. Due to the pressure differential, the air then moves through the permeable top layer 10 into the external airspace 23. The air exiting the top layer 10 is at a much lower pressure and velocity in the air exiting from the lower airspace 22 to the upper airspace 21 through middle layer 11. Accordingly, the turbulence of the air exiting through top layer 10 is less than the turbulence of the air exiting through middle layer 11. Thus, the air exiting top layer 10 toward the patient is much less turbulent than the air exiting the middle layer 11. The temperature regulating pad 1, therefore, is effective in reducing the air turbulence around the patient thereby minimizing the production and movement of dust in the operating room while still adequately maintaining or increasing the body temperature of the patient. In that sense, any residual dust inside the pad 1 from manufacturing or storage is less likely to be pushed through and out of the pad 1 and into the surgical environment. In some embodiments, therefore, the top layer 10 is air-permeable to function as an air filter to permit the flow of air out but prevent or significantly limit the flow of dust and other particulates. FIG. 6 shows an embodiment of the temperature regulating pad 1 showing the various airspaces with an exemplary patient 40 disposed thereon and subjected to the air flow that the pad 1 herein provides. In some embodiments, the forced air is relatively warm in order to regulate the patient's body temperature and prevent or alleviate hypothermia. In other embodiments, the forced air may be relatively cool in order to reduce the patient's body temperature in the case of hyperthermia or other scenarios where it is desirous to reduce the patient's body temperature.

As noted above, certain materials may be selected for each of the top layer 10, middle layer 11, and bottom layer 12 to promote the functionality of the pad 1. For example, the top layer 10 may comprise a comfortable material such as polypropylene that is air permeable and breathable to permit air to pass through but prevents or limits the passage of dust or other particulates. In some embodiments, the top layer 10 is permeable to air but prevents or substantially reduces the passage of dust or other particulate, in effect acting as an air filter. The middle layer in some embodiments comprises a thin plastic that includes the aforementioned apertures 111 and also permits for seal lines to be created between it and the bottom layer 12, which seal lines 14 may be constructed by heat sealing or other known construction methodology. In some embodiments bottom layer 12 comprises an impermeable low friction material such as nylon to facilitate movement of a patient with respect to an underlying support surface. It is appreciated and understood that the temperature regulating pad 1 is generally suitable for regulating the temperature of a patient and need not necessarily be used with warm air. In certain applications, the pad 1 could be equally used with forced cool air and would function substantially as explained and described herein.

The temperature regulating pad 1, in some embodiments comprises an air-permeable polypropylene top layer 10, a perforated plastic middle layer 11, and an impermeable low friction nylon bottom layer 12. The perforated middle layer 11 is disposed between the top layer 10 and the bottom layer 12 and is at least partially attached to the bottom layer by a plurality of spaced apart seal lines 14. The top layer 10 and the perforated middle layer 11 delimit an upper airspace 21. The perforated middle layer 11 and the bottom layer 12 delimit a lower airspace 22. Forced air of a desired temperature is received into the lower airspace 22 and flows from the lower airspace 22 into the upper airspace 21 through the perforated middle layer 11. The forced air exits the air-permeable top layer 10 into an external airspace 23 about said top layer 10 to regulate the temperature of a patient 40 resting on the pad. The air exiting the air-permeable top layer 10 into the external airspace 23 is less turbulent than the air flowing from the lower airspace 22 to the upper airspace 21 to limit the excitement of dust and particulate in and around the area surrounding the pad.

While specific embodiments have been described in detail in the foregoing detailed description and illustrated in the accompanying drawings, those with ordinary skill in the art will appreciate that various modifications and alternatives to those details could be developed in light of the overall teaching of the disclosure. Accordingly, the particular arrangements disclosed are meant to be illustrative only and not limiting as to the scope of the invention, which is to be given the full breadth of the appended claims in any and all equivalents thereof.

What is claimed is:

1. A temperature regulating pad, comprising:
an air-permeable top layer, a perforated middle layer, and a bottom layer, said perforated middle layer disposed between said top layer and said bottom layer;
said top layer and said perforated middle layer delimiting an upper airspace and said perforated middle layer and said bottom layer delimiting a lower airspace; and
wherein forced air is received into said lower airspace and flows from said lower airspace into said upper airspace through said perforated middle layer, exiting said air-permeable top layer into an external airspace about said top layer.

2. The temperature regulation pad of claim 1, wherein said air exiting said air-permeable top layer into said external airspace is less turbulent than said air flowing from said lower airspace to said upper airspace.

3. The temperature regulation pad of claim 1, wherein said perforated middle layer is at least partially attached to said bottom layer.

4. The temperature regulation pad of claim 3, wherein said perforated middle layer is at least partially attached to said bottom layer by a plurality of spaced apart seal lines.

5. The temperature regulation pad of claim 1, wherein said plurality of spaced apart seal lines delimit one or more longitudinal channels within said lower airspace.

6. The temperature regulating pad of claim 1, further comprising an inlet in flow communication with said lower airspace, wherein said inlet receives said forced warm air.

7. The temperature regulation pad of claim 1, wherein said perforated middle layer includes a perforated center section surrounded by an impermeable border.

8. The temperature regulation pad of claim 1, wherein said bottom layer comprises a low friction nylon material.

9. The temperature regulation pad of claim 1, wherein said middle layer comprises plastic.

10. The temperature regulation pad of claim 1, wherein said top layer comprises polypropylene.

11. A temperature regulating pad, comprising:
an air-permeable polypropylene top layer, a perforated plastic middle layer, and a low friction nylon bottom layer, said perforated middle layer disposed between said top layer and said bottom layer;
said top layer and said perforated middle layer delimiting an upper airspace and said perforated middle layer and said bottom layer delimiting a lower airspace;
wherein forced air is received into said lower airspace and flows from said lower airspace into said upper airspace through said perforated middle layer, exiting said air-permeable top layer into an external airspace about said top layer; and
wherein said air exiting said air-permeable top layer into said external airspace is less turbulent than said air flowing from said lower airspace to said upper airspace.

12. The temperature regulation pad of claim 11, wherein said perforated middle layer is at least partially attached to said bottom layer.

13. The temperature regulation pad of claim 12, wherein said perforated middle layer is at least partially attached to said bottom layer by a plurality of spaced apart seal lines.

14. The temperature regulation pad of claim 11, wherein said plurality of spaced apart seal lines delimit one or more longitudinal channels within said lower airspace.

15. The temperature regulating pad of claim 11, further comprising an inlet in flow communication with said lower airspace, wherein said inlet receives said forced warm air.

16. The temperature regulation pad of claim 11, wherein said perforated middle layer includes a perforated center section surrounded by an impermeable border.

17. A temperature regulating pad, comprising:
an air-permeable polypropylene top layer, a perforated plastic middle layer, and an impermeable low friction nylon bottom layer, said perforated middle layer disposed between said top layer and said bottom layer;
said perforated middle layer at least partially attached to said bottom layer by a plurality of spaced apart seal lines;
said top layer and said perforated middle layer delimiting an upper airspace and said perforated middle layer and said bottom layer delimiting a lower airspace;
wherein forced air is received into said lower airspace and flows from said lower airspace into said upper airspace through said perforated middle layer, exiting said air-permeable top layer into an external airspace about said top layer; and
wherein said air exiting said air-permeable top layer into said external airspace is less turbulent than said air flowing from said lower airspace to said upper airspace.

18. The temperature regulation pad of claim 17, wherein said plurality of spaced apart seal lines delimit one or more longitudinal channels within said lower airspace, said longitudinal channels having an substantially semi-cylindrical shape when inflated by said forced air.

19. The temperature regulation pad of claim 17, wherein said perforated middle layer includes a perforated center section surrounded by an impermeable border.

20. The temperature regulation pad of claim 17, further including an oversheet configured to surround said external airspace.
